(19) United States
(12) Patent Application Publication
(10) Pub. No.: US 2023/0400463 A1
(43) Pub. Date: Dec. 14, 2023

(54) RAPID DIAGNOSTICS USING PHASE COUPLING OF ANTIGENS

(71) Applicant: Udo Riß, Ratzeburg (DE)

(72) Inventor: Udo Riß, Ratzeburg (DE)

(21) Appl. No.: 18/033,118

(22) PCT Filed: Oct. 20, 2021
(86) PCT No.: PCT/DE2021/200164

§ 371 Date: Apr. 21, 2023

(30) Foreign Application Priority Data
Oct. 25, 2020 (DE) 10 2020 006 525.2

(51) Int. Cl. G01N 33/569; G01N 33/543

(52) U.S. Cl. CPC G01N 33/56983; G01N 33/54386; G01N 2333/165

(57) ABSTRACT

A method is provided with which biomolecules (5) such as viruses, for example, are quantitatively determined and/or separated very rapidly. Antigens or receptors (2) are directly or indirectly fixed so closely together on a substrate (1) that they couple together in phase. A dynamic charge displacement field is significantly increased compared to a single antigen or receptor (2) by means of the phase coupling, so that biomolecules (5) which are located in the range of the larger charge displacement field move immediately in the direction of the phase-coupled antigens or receptors (2). The binding kinetics are significantly faster than in the case of conventional methods. The invention enables, for example, the construction of a rapid test for coronavirus which can deliver a result within seconds. The sensitivity which can be obtained is that of one virus. The method can be applied to liquids (6) or aerosols (7).

### CROSS REFERENCE TO RELATED APPLICATIONS

This application is a United States National Phase Application of International Application PCT/DE2021/200164, filed Oct. 22, 2021, and claims the benefit of priority under 35 U.S.C. § 119 of German Application 10 2020 006 525.2, filed Oct. 25, 2020, the entire contents of which are incorporated herein by reference.

### TECHNICAL FIELD

The invention relates to a method with which biomolecules such as enzymes, interleukins, chemokines, neurotransmitters, antibodies, viruses, bacteria and cells can be detected and quantified within seconds. The sensitivity of the method is in the single-molecule range. In addition, the invention relates to an analytical means and an analytical device for carrying out the method.

An important application is in a rapid test, with which SARS-CoV-2 viruses in exhaled respiratory air or in liquids such as blood serum can be assayed within a few seconds.

### BACKGROUND

For antibody and virus diagnostics, methods are known with which it is possible to detect the presence of antibodies in a liquid. Examples of these methods are ELISA and RIA, as well as the rapid SARS-CoV-2 antigen test with gold particles. However, all of these methods require labels in the form of enzymes, gold particles or radioactivity. The labelling reduces the speed of the measurement so much that it can no longer be considered to be a rapid test; as an example, the rapid SARS-CoV-2 antigen test requires approximately 15 minutes before a result can be obtained. In addition, the sensitivity is not especially high; in the case of the rapid SARS-CoV-2 antigen test, the sensitivity can be considered to be approximately 3.2 million viruses. This means that 60% of all coronavirus infections cannot be detected. A further problem is quantification—because of method-related errors, only very coarse quantification is possible. In the case of the rapid SARS-CoV-2 antigen test, therefore, quantification is dispensed with entirely and only a qualitative “infected” or “not infected” distinction is provided.

In the case of viral analysis, the “gold standard” is the PCR test. In theory, a single virus can be detected, but in practice, a best case sensitivity of between 100 and 1000 viruses can be assumed. According to the Deutsches Ärzteblatt [German Medical Journal] 24/2020¹, the specificity of the coronavirus test is about 95%, meaning that the specificity is significantly lower than the specificity of rapid antigen tests, with 99.81%. According to the Medical Journal 24/2020, the probability of error for a false positive result is a prevalence of 3% at 70%. In the case of a prevalence of 80%, the probability of a false negative result is 56%. In addition, the laboratory-based PCR technique requires several hours for the analysis. People who have been tested frequently have to wait 1-2 days before they receive a result. The most rapid PCR tests which are currently on the market have a measurement time of 39 minutes. However, this short measurement time comes at the cost of the measurement sensitivity. Quantification with PCR is very limited; often, only a qualitative distinction can be made between “not infected”, “low viral load” and “high viral load”. The limits in this regard are 100000 and 1000000 virus copies/mL. The World Health Organization, WHO, indicates that the PCR technique is extremely sensitive to errors and reliable results can only be obtained if they are carried out extremely carefully.

Ben-Gurion University has developed a one-minute coronavirus test² which is based on THz spectroscopy. This test costs approximately $50 per measurement and is said to have a specificity of approximately 90%. However, this test is not yet at the production stage.

A similar method based on THz time domain spectroscopy has been developed by the Tera Group. The sensitivity of the method is approximately 4 viruses/μm². Because of the large bandwidth of the method, the specificity should be small. The large bandwidth also has a negative influence on the cross-reactivity as regards similar diseases.

Other technologies are being developed; they concern the analysis of VOCs (volatile organic compounds) and Raman spectroscopy. However, as yet, neither technology has been fully developed.

It is known that enzymes exhibit burst kinetics. In the burst kinetics zone, enzymes couple to their substrates particularly rapidly. Metrological experiments carried out by us have shown that antibodies, viruses, bacteria and cells can also exhibit burst kinetics. In some interleukins, neurotransmitters and chemokines, chemical degradation is also self-evident. A publication with measurement curves³ has been issued in the case of antibodies. The linear portion of the measurement curve in FIG. 3 exhibits the burst kinetics.

However, these burst kinetics are usually not visible with conventional substrate materials (polystyrene, nitrocellulose) for immunological tests, because the occupation density produced with antigens on these materials is too low. In the case of nitrocellulose, antigens dock only in the pores, and the pore density is not usually below a value of 600 nm. In the case of polystyrene, docking only takes place in the irregularities. In the case of polystyrene, the antigen density can be increased by irradiation, but even then the antigen density is usually still not high enough for clearly visible burst kinetics to be obtained. If antigens are bound onto conventional substrates, then in addition, partial desorption of antigens has to be anticipated, i.e. antigens leave the substrate surface or biomolecules coupled to ligands or antigens are torn from the surface. This reduces the accuracy and reproducibility of the known methods. As a rule, the conventional substrates are provided with a recommended antigen concentration. This antigen concentration has been empirically determined and is used in the majority of antigen-based tests.

The interaction of antibody layers and membranes covered with antibodies and antigens is also known to occur.

### SUMMARY

In accordance with the invention, there are two different methods for generating significant burst kinetics, namely phase coupling of antigens via their charges and mechanical phase coupling. These significant burst kinetics have been measured empirically here on substrates in accordance with the invention with an ellipsometer. While ellipsometer measurements on conventional substrates such as polystyrene or nitrocellulose slides produce no visible burst kinetics, on the substrates in accordance with the invention, two observations which differ fundamentally from the prior art have been made. On the one hand, the burst kinetics are clearly visible as a straight characteristic line; on the other hand, all biomolecules (antibodies, viruses, etc.) couple to the antigen-coated substrates if the test cell is selected so that it is small enough. If the liquid is removed from this test cell and the experiment is started afresh with a fresh antigen-coated substrate carrier, then no molecules couple to the substrate carrier, leading to the conclusion that there are no more biomolecules in the liquid. It can generally be assumed that biomolecules vibrate in the terahertz frequency range and that they have electric charges in the form of NH₃+ and COO— ions. According to Maxwell's equations, vibrating charges produce electric charge displacement waves. They are represented by the letter D in Maxwell's equations. Each individual biomolecule produces its own electric charge displacement field. The invention is based on phase coupling via charges on a novel type of substrate material. These are materials which oxidize readily and in addition are extremely flat. They may, for example, be smooth silicon or aluminum substrates. In both materials, an oxide layer is rapidly formed. In the case of silicon, the SiO²⁻ oxide layer grows by ca. 0.7 nm in the first hour. In the case of aluminum, automatic oxidation occurs.

Silicon wants its outer shell, which contains 4 electrons, to be occupied; this is similar in the case of aluminum, which has 3 valence electrons in the outer shell. Other materials are also possible.

Because of the incomplete occupation of the outer shell, the substrate materials endeavor to fill the outer shell, which happens in air by the oxidation process. Alternatively, the COO— ions of antigens or antibodies can bind to the substrates. In this regard, the molecules bind to the substrate in identical orientations, i.e. the molecules all have an identical orientation. In accordance with the invention, the molecules can then couple in phase.

Thus, in accordance with a first aspect of the present invention, a method is provided for the quantitative determination and/or separation of biomolecules which is characterized in that a plurality of antigens or receptors each with at least one alpha helix or a beta sheet are directly or indirectly fixed on a substrate so closely together that their vibrations couple together in phase via electric charges or mechanically, wherein an electric charge displacement field of the plurality of antigens or receptors which are coupled in phase is formed which is larger compared to an electric charge displacement field of an individual antigen or receptor, wherein the biomolecules in the electric charge displacement field of the plurality of antigens or receptors which are coupled in phase couple to the antigens or receptors specifically and rapidly.

Optionally, the biomolecules may respectively contain at least two alpha helices or two beta sheets.

Optionally, antibodies which specifically bind to the biomolecules may be used as the antigens or receptors.

Optionally, a liquid layer containing the biomolecules may be formed above the substrate with the plurality of antigens or receptors which are coupled in phase, wherein the liquid layer has a layer thickness up to a maximum layer thickness at which all or the predominant fraction of the biomolecules in the liquid couple to the antigens or receptors.

Optionally, the biomolecules may be present in an ambient air or respiratory air aerosol, wherein the ambient air or respiratory air aerosol is guided over the substrate with the plurality of antigens or receptors which are coupled in phase, and wherein the temperature of the substrate with the plurality of antigens or receptors which are coupled in phase is controlled in a manner such that condensation of the ambient air or respiratory air aerosol occurs on it.

Optionally, the method may be used to quantitatively determine and/or to separate the biomolecules (for example neurotransmitters, interleukins, chemokines, enzymes, antibodies, viruses, bacteria, or cells) from circulating blood, from a cerebrospinal fluid or from respiratory air from a human being or animal in vitro.

Optionally, the biomolecules may be in a liquid or in an ambient air or respiratory air aerosol which is guided in a defined continuous flow process or throughflow process over the substrate with the plurality of antigens or receptors which are coupled in phase.

Optionally, an oxidizing material (for example silicon or aluminum) may be used as the substrate and blocking of regions of the substrate which are not occupied by antigens or receptors is carried out by means of oxidation or by means of other organic or inorganic materials.

Optionally, regions of the substrate which are not occupied by antigens or receptors may be oxidized and an oxide layer which is formed thereby is rendered hydrophobic by a natural or synthetic coating.

Optionally, a linear or elliptically polarized laser beam may be directed onto the substrate with the plurality of antigens or receptors which are coupled in phase with the coupled biomolecules and a laser beam which is reflected therefrom is detected in a detector.

Optionally, a camera may be used as the detector.

Optionally, scattered light produced from the biomolecules coupled to the antigens or receptors may be detected and analyzed for the quantitative determination of the number of coupled and therefore separated biomolecules.

Optionally, the substrate with the plurality of antigens or receptors which are coupled in phase may be used multiple times for the quantitative determination and/or separation of biomolecules from different liquids or ambient air or respiratory air aerosols until a relationship between the number of biomolecules coupled to the antigens or receptors and the scattered light produced therefrom is no longer linear.

Optionally, for successive quantitative determinations and/or separations of biomolecules using the same substrate and the same plurality of antigens or receptors which are coupled in phase, the variation in the scattered light produced with respect to the previous quantitative determination and/or separation is analyzed for the quantitative determination of the number of coupled and therefore separated biomolecules. Depending on the biomolecule load per application, with each application, a layer thickness of biomolecules coupled to the antigens or receptors increases. Because of the larger charge displacement field of the plurality of antigens or receptors which are coupled in phase, the biomolecules are also coupled via intermediate layers of biomolecules which are already coupled together. Thus, several layers of biomolecules may be generated. In the case of a high viral load, this may happen with a single application. As long as there is a linear relationship between the scattered light and the number of coupled biomolecules, the substrate can be used for further applications. Beyond a certain layer thickness of coupled biomolecules, however a linear relationship is no longer guaranteed and the substrate must be changed. It should be noted in this regard that a linear relationship does not require whole biomolecule layers to be completely full. Even in the case of only partially filled layers of biomolecules, there is a linear relationship between the scattered light and the number of coupled biomolecules wherein, for example, the scattered light of an only half-filled layer of biomolecules corresponds to the scattered light of a virtually completely filled layer of biomolecules with half the layer thickness. When the irradiated surface area of the substrate is known and the size of the biomolecules is known, therefore, the relationship can easily be calculated. In point of fact, even individually coupled biomolecules vary the scattered light to the same extent as a virtual biomolecule layer extending over the entire irradiated surface area with the volume of this single coupled biomolecule, i.e. a very small sub-molecular “layer thickness”.

Optionally, the plurality of antigens or receptors may be indirectly fixed to the substrate such that the plurality of antigens or receptors is applied to an elastic or rigid membrane (for example a lipid membrane or a cell membrane or virus membrane with epitopes) and the membrane is floated onto the substrate on a layer of water. Preferably, the layer of water is a monolayer of water forming automatically between the substrate and the membrane.

Optionally, the method may furthermore comprise a step for the qualitative assessment of a biomolecule load based on the quantitatively determined number of coupled biomolecules compared with a specified reference value. As an example, the qualitative assessment of a biomolecule load may be such that when a specified reference value is exceeded, the qualitative assessment “infected” is issued and/or when a specified reference value is not reached, the “not infected” assessment is issued. In this regard, gradations defined by several reference values may be provided such as, for example, “not infectious”, “slightly infectious”, “moderately infectious” or “highly infectious”.

In accordance with a second aspect of the present invention, an analytical means is provided for the quantitative determination and/or separation of biomolecules, comprising: a substrate and a plurality of antigens or receptors each with at least one alpha helix or a beta sheet. In this regard, the plurality of antigens or receptors is directly or indirectly fixed on the substrate so closely together that their vibrations are coupled together in phase via electric charges or mechanically, wherein a larger electric charge displacement field of the plurality of antigens or receptors which are coupled in phase is formed compared to an electric charge displacement field of a single antigen or receptor, wherein the larger electric charge displacement field of the plurality of antigens or receptors which are coupled in phase can be used to couple biomolecules to the antigens or receptors specifically and rapidly. The analytical means may preferably be a single-use or multi-use consumable which can be packaged, stored and transported in the dry state. Preferably, the analytical means has a surface area of less than 25 cm² or less than 9 cm².

Optionally, the antigens or receptors may be antibodies which specifically bind to the biomolecules.

Optionally, the temperature of the substrate with the plurality of antigens or receptors which are coupled in phase can be controlled in a manner such that an ambient air or respiratory air aerosol containing the biomolecules condenses on it.

Optionally, the substrate may comprise an oxidizing material such as silicon or aluminum, for example, and regions of the substrate which are not occupied by antigens or receptors may be occupied by means of oxidation or by another organic or inorganic material.

Optionally, regions of the substrate which are not occupied by antigens or receptors may have an oxide layer which is hydrophobic because of a natural or synthetic coating.

Optionally, the plurality of antigens or receptors may be indirectly fixed on the substrate in a manner such that the plurality of antigens or receptors is applied to an elastic or rigid membrane (for example a lipid membrane or a cell membrane or virus membrane with epitopes) and the membrane is floated onto the substrate on a layer of water. Preferably, the layer of water is a monolayer of water which forms automatically between the substrate and the membrane.

In accordance with a third aspect of the present invention, an analytical device for the quantitative determination and/or separation of biomolecules in a liquid or an ambient air or a respiratory air aerosol is provided, wherein the analytical device comprises:
an analytical means as described above,
a continuous flow or throughflow device for guiding the liquid or the ambient air or respiratory air aerosol over the analytical means,
a laser beam decoupler for decoupling a linear or elliptically polarized laser beam onto the analytical means, and
a detector for detecting a laser beam reflected from the analytical means.

Optionally, the analytical means may be disposed in the analytical device so that it can be changed after a single use or after multiple uses.

Optionally, the detector may be a camera.

Optionally, the analytical device may furthermore comprise an analysis unit which is equipped and configured to analyze scattered light produced from the biomolecules coupled to the antigens or receptors of the analytical means, which is detected by the detector for the quantitative determination of the number of coupled biomolecules.

Optionally, the analysis unit may be further equipped and configured to qualitatively assess a biomolecule load based on the quantitatively determined number of coupled biomolecules in comparison to a specified reference value. As an example, the qualitative assessment of a biomolecule load may be such that when a specific reference value is exceeded, the qualitative assessment “infected” is issued, and/or when a specific reference value is not reached, the “not infected” assessment is issued. In this regard, gradations defined by several reference values may be provided, such as “not infectious”, “slightly infectious”, “moderately infectious” or “highly infectious”.

Optionally, the analytical device may further comprise a temperature control unit which is equipped and configured to control the temperature of the analytical means so that an ambient air or respiratory air aerosol containing the biomolecules condenses on it.

Exemplary embodiments of the invention are shown in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 and FIG. 1b illustrate exemplary embodiments of the electrical phase coupling;

FIG. 1c and FIG. 1d illustrate exemplary embodiments for the mechanical phase coupling; and

FIG. 2a and FIG. 2b are schematic views illustrating method aspects.

### DESCRIPTION OF PREFERRED EMBODIMENTS

Exemplary Production of Electrical Phase Coupling with Blocking:

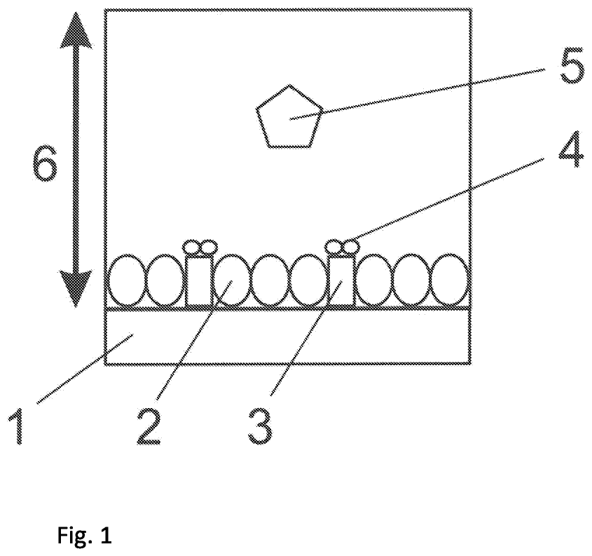

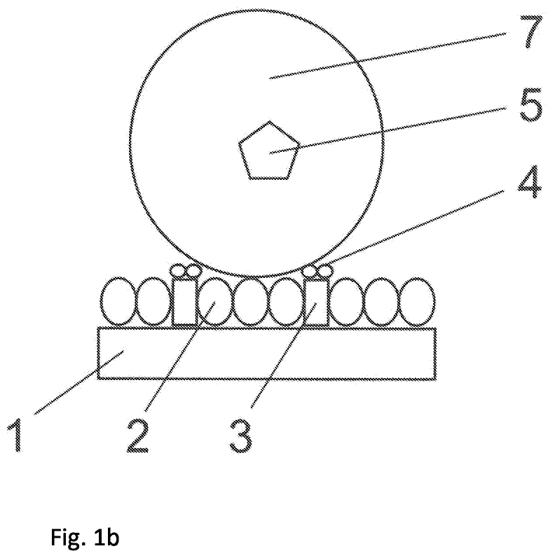

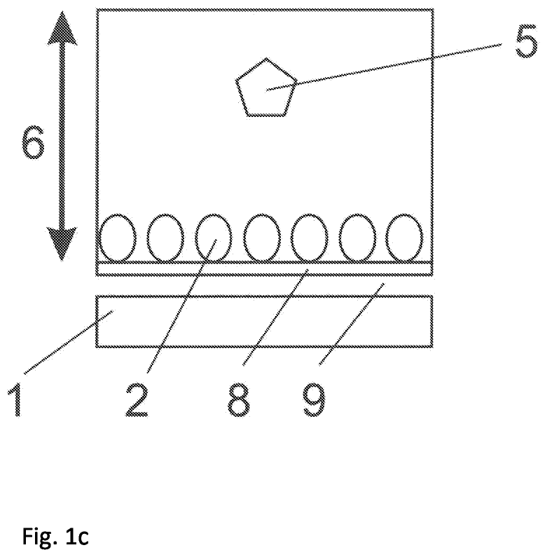

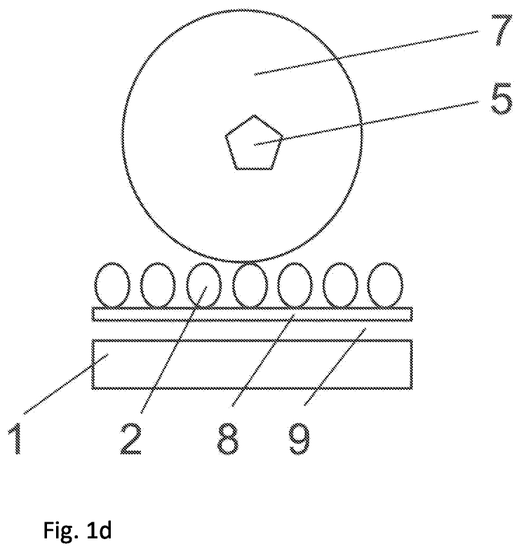

In the case of electrical phase coupling (FIGS. 1 and 1b), a substrate (1) is initially rendered free from oxide or the oxide layer is significantly reduced in order to couple antigens or receptors (2). In the case of antigens, these may also be complete antibodies which specifically bind to biomolecules (5) which are to be quantitatively determined and/or separated.

Oxide-free or oxide-reduced silicon may, for example, be obtained by freeing a silicon wafer with a SiO₂ layer from its oxide layer using hydrofluoric acid. Application of antigens or antibodies in aqueous solution to the oxide-free or oxide-reduced silicon wafer (1) leads to coupling of the antigens or antibodies (2), so that these sit securely on the silicon wafer and are orientated in the same direction.

In the case of aluminium, aluminium foil may be used as the substrate (1). During the production process, the foil is rolled in the folded state, wherein the inside of the folded foil is substantially free from oxide. Oxidation only begins when the foil is pulled apart. If antigens or antibodies in aqueous solution are applied to the foils at this point in time, then they adhere immediately.

In order to obtain the phase coupling via charges, the mutual distance between the antigens or antibodies (2) must be so small that the charges of adjacent antigens or antibodies (2) can interact with each other.

Antigens (2) have positive charges in the form of NH₃⁺ ions and negative charges in the form of COO⁻ ions; the isoelectric point is between the positive and the negative charges. In addition, antigens have at least one beta sheet or alpha helix. It is generally known that both beta sheets as well as alpha helices behave like linear or non-linear springs. It is also generally known that the structures vibrate or can vibrate in the terahertz range. For this, the structures can only be moist or in aqueous solution.

By applying the mathematical methods of engineering mechanics and using Maxwell's equations, it can be shown that automatic phase coupling of the antigen vibrations occurs when the antigens (2) are sufficiently densely disposed adjacent to each other.

For an antigen distance of 3 nm, phase coupling occurs via electric charges in all cases. This can readily be demonstrated mathematically using the two adjacent mass-spring-mass system model, wherein both masses are electrically charged. The two mass-spring-mass systems synchronise independently of the starting conditions with time. The only prerequisite is that both mass-spring-mass systems have the same resonance frequency.

Phase coupling is particularly strong when a flat plane can pass through all of the isoelectric points of the antigens (2). Thus, the substrate (1) should be as smooth as possible. However, phase coupling also occurs when a flat plane cannot be passed through the isoelectric points. An increase in the sensitivity of the method may also be obtained when several antigen layers or other layers are applied. This additional layer is preferably to be introduced between the substrate (1) and the antigen layer (2).

By overlaying of the individual fields, the antigens (2) which are fixed and which vibrate in phase produce a resultant electric charge displacement field which is larger than the respective charge displacement field of an individual antigen (2). The range of the charge displacement waves increases with the root of the number of coupled antigens (2). This can be calculated with conventional electrical engineering methods. For a rapid test, it is therefore necessary for sufficient antigens (2) to be coupled in phase.

Experiments have shown: if whole antibodies (2) are used instead of simple antigens, then the burst kinetics of ligands or biomolecules (5) accelerate significantly.

After producing an antigen or antibody layer which is not necessarily complete, blocking of unoccupied regions of the substrate (1) is carried out by means of molecules (3) or by means of the natural oxidation process (3). Natural blocking by oxidation can be carried out via a natural or synthetic oxidation process. In the case of a natural oxidation process, the natural oxygen of the air is used; in the case of a synthetic oxidation process, additional oxygen is supplied. Unfortunately, SiO₂ is hydrophilic, but it can be made hydrophobic. To this end, the SiO₂ layer can simply be exposed to normal air. After a certain time, a hydrophobic layer (4) is formed on the SiO₂. This process can be shortened by vaporizing oil or fat in the vicinity. An extremely thin hydrophobic film then forms on the SiO₂. This extremely thin hydrophobic film improves blocking of unoccupied regions of the substrate (1).

As an alternative to SiO₂ blocking, blocking with other substrates may also be carried out. Possible examples in this regard are lipids or TRIS. These substrates have the task of preventing non-specific binding to regions of the substrate (1) which are not occupied by antigens or receptors (2).

### Exemplary Embodiment for Mechanical Phase Coupling:

As an alternative or in addition to coupling via charges, as described above, mechanical phase coupling of the antigens or antibodies may occur. An example of mechanical phase coupling is illustrated in FIGS. 1c and 1d.

Antigens or antibodies (2) on an elastic or rigid membrane (8) are placed on a substrate (1) with or without an oxide layer (3) and which is as smooth as possible (for example a silicon substrate). The membrane may, for example, be a cell membrane or virus membrane with epitopes. A monolayer of water (9) automatically forms between the lipid membrane (8) and the substrate surface (1); the monolayer enables the membrane to vibrate, and so mechanical phase coupling of the antigens can occur.

The mechanical phase coupling of metronomes which are positioned on a movable plate is known. This principle also applies to biomolecules.

In the case of mechanical phase coupling as described above, blocking can be dispensed with, because lipid membranes make suitable blockers. In order to obtain mechanical phase coupling, the distance between the antigens or antibodies (2) may be larger compared to electrical phase coupling, because the antigens or antibodies (2) are mechanically coupled by means of the membrane (8).

Exemplary Embodiment with Electrical or Mechanical Phase Coupling

The rest of the procedure is largely the same, irrespective of the phase coupling (mechanical or electrical).

Above the antigens or antibodies (2) is a liquid layer (6) with the biomolecules (5) to be analysed. The liquid layer (6) is, however, only thick enough to allow the electric charge displacement waves to penetrate with sufficient strength. The field strength here is large enough for the biomolecules (5), for example enzymes, antibodies, viruses, bacteria or cells, to be recognised at any location in the liquid layer (6) where antigens or antibodies (2) are found. They will then automatically move in the direction of the antigens or antibodies in order to couple to them. The thinner the layer, the faster will the coupling process occur. If the aforementioned general conditions are satisfied, then all of the biomolecules (5) will couple, i.e. there is a significant deviation from the law of mass action. This has been shown in experiments. The non-linear kinetics of the law of mass action are replaced by a controlled process with linear kinetics. This controlled process is significantly faster (by powers of ten) than the law of mass action and leads to the development of rapid tests.

Experimentally, a region of approximately 2 mm could be identified in which biomolecules (5) such as viruses recognise their antigens or antibodies (2) and move towards them; in the case of bacteria and cells, something similar can be expected. In theory, this region could be extended to more than 10 mm by optimization of the receptor-layer system.

It has also been shown experimentally that in this region, the biomolecules move towards the substrate at a constant speed. In experiments with IgE antibodies, a speed of 2 mm/s was determined; with viruses, the speed was 0.1-0.2 mm/s.

In the case of a column of liquid (6) of 1 mm above the antigen or antibody-coated substrate (2), all biomolecules in the form of IgE antibodies are deposited from the liquid column within 0.5 seconds. In the case of viruses, the time is ca. 5-10 seconds. Instead of a continuous liquid column (6), aerosols (7 in FIGS. 1b and 1d), in which biomolecules (5) such as viruses are located, are measured. In the case of a diameter for the aerosols (7) of 1 micrometre, viruses require a time period of 5 to 10 milliseconds for coupling.

In order to detect aerosols (7) metrologically, the substrate (1) should be colder than the aerosol (7) or be below the dew point. This means that the aerosols (7) will condense on the antigen layer or antibody layer (2) and the coupling process will occur.

The metrological evaluation may, for example, be carried out in accordance with U.S. Pat. No. 6,168,921, but since then, other further developments based on this have been developed which have a significantly higher sensitivity. With the aid of this technology, a variation in layer thickness of 20 femtometres can be detected. The individual biomolecules (5) are in fact larger, but the optical diffraction limit means that mean virtual layer thicknesses which are significantly below one atomic diameter can be measured. If a 160 nm virus with a volume of 2.1*10⁻³ μm³ is coupled to a measurement surface area of 3 mm², then this corresponds to a mean virtual layer thickness increase of 0.71 femtometres on the 3 mm² substrate surface. In the case of an instrumentational engineering resolution, then the method has a resolution of 28 viruses (5), for example.

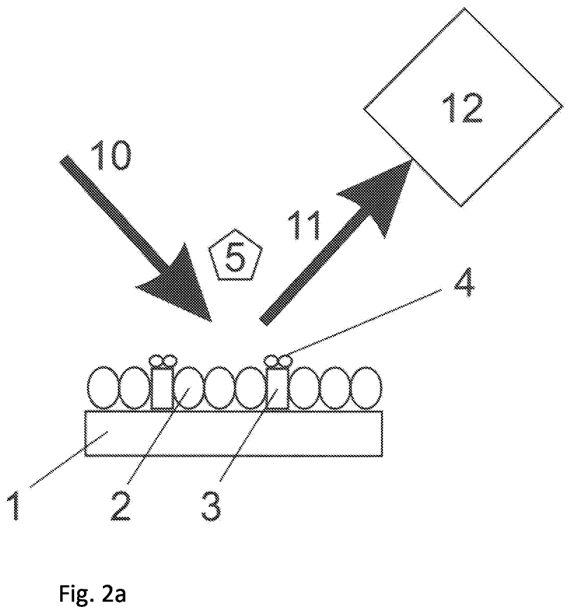

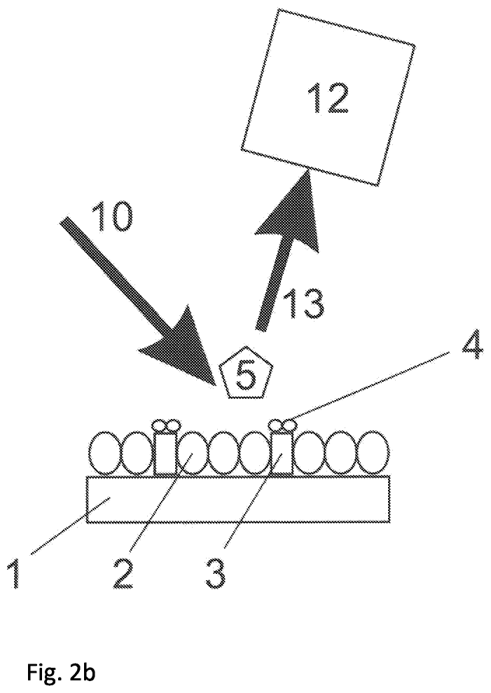

Using a camera as the detector (12) means that the resolution can be increased still further. Even if the signal noise of the camera (12) is a factor of 10000 higher than in the case of the method without a camera and the layer thickness resolution of each pixel is only 0.2 nm, in the case of a diffraction limit (lateral resolution of the camera) of 500 nm, then even an individual virus (5) can be identified really well. The resolution limit of the method is then approximately 1/10 viruses. FIG. 2a illustrates this method. A linear or elliptically polarised laser beam (10) is reflected, the reflected beam (11) reaches a camera (12) with or without a polarisation filter.

If the substrate (1) is very smooth, then the antigen or antibody layer (2) on the substrate (1) does not produce any significant scattered light. If larger biomolecules (5) such as viruses, bacteria or cells are coupled to the antigens or antibodies (2), then this produces scattered light (13). Even biomolecules (5) which are smaller than the diffraction limit produce scattered light (13) which can be seen with the naked eye or with optical aids (for example imaging optics with a camera).

Experiments have shown that even 40 nm biomolecules (5) can be seen with the naked eye. This is possible because the scattered light (13) has a diverging beam path. FIG. 2b shows the method by way of an example. A laser beam (10) produces scattered light (13) when it impinges on the coupled biomolecule (5). This is detected either with the eye or with a camera (12). Introducing polarised or anisotropic elements into the paths of the beam (10, 13) can even more significantly increase the signal-to-noise ratio.

As an alternative and independently, the present invention may also be defined as follows:

A method for detecting, removing or filtering out molecules is provided, characterized in that antigens or receptors with at least one molecular spring (alpha helix or beta sheet) are fixed on a substrate, the vibrations being coupled in phase via electric charges or mechanically (for example lipid membranes) in order to produce a larger electric charge displacement field, wherein the larger range of the field is used to bring ligands to their receptors in a specific and rapid manner.

Optionally, the ligands may have at least two springs (alpha helices or two beta sheets).

Optionally, antibodies may be used as the antigens or receptors.

Optionally, the thickness of the liquid layer above the substrate with the phase-coupled antigens may be selected in a manner such that all or the predominant fraction of the ligands couple to the antigens in the liquid.

Optionally, ligands in ambient air or respiratory air aerosols may be determined, removed or filtered out, wherein the temperature of the substrate with the receptors is controlled in a manner such that condensation of the aerosol on the substrate occurs.

Optionally, the method may be used to remove biomolecules (neurotransmitters, interleukins, chemokines, enzymes, antibodies, viruses, bacteria or cells) from circulating blood, from cerebrospinal fluid or from respiratory air.

Optionally, the liquids or aerosol-containing gases (for example ambient air) may involve a continuous flow or throughflow method.

Optionally, oxidising materials (for example silicon or aluminium) may be used as the substrate and blocking may be carried out by oxidation or using other organic or inorganic materials.

Optionally, the oxide layers may be rendered hydrophobic by natural contamination or synthetic coating.

Optionally, the measurement may be carried out optically or mechanically with the aid of a cantilever.

Optionally, a camera may be used for detection in order to increase the resolution of the method.

Optionally, the scattered light may be detected with a camera.

While specific embodiments of the invention have been shown and described in detail to illustrate the application of the principles of the invention, it will be understood that the invention may be embodied otherwise without departing from such principles.

### REFERENCES TO LITERATURE

1. Deutsches Arzteblatt [German Medical Journal] 24/2020, issue A, page 1194 ff.
2. American Association for the Advancement of Science (AAAS), News Release 27 May 2020, “One minute electro-optical coronavirus test developed at Ben-Gurion University”, https://www.eurekalertorg/news-releases/497554
3. Riβ, Udo, (2011/06/30), “Theory of long distance interaction between antibodies and antigens”, European Biophysics Journal: EBJ. 40. 987-1005. 10.1007/s00249-011-0718-z.
4. Alexandre Rothen, “IMMUNOLOGICAL REACTIONS BETWEEN FILMS OF ANTIGEN AND ANTIBODY MOLECULES”, Journal of Biological Chemistry, Volume 168, Issue 1, 1947, Pages 75-97, ISSN 0021-9258, https://doi.org/10.1016/S0021-9258(17)35094-9. (https://www.sciencedirect.com/science/article/pii/S0021925817350949)

1. A method for the quantitative determination and/or separation of biomolecules, the method comprising the step of fixing a plurality of antigens or receptors, each with at least one alpha helix or a beta sheet, directly or indirectly on a substrate so closely together that vibrations of the plurality of antigens or receptors couple together in phase via electric charges or mechanically, wherein an electric charge displacement field of the plurality of antigens or receptors which are coupled in phase is formed which is larger compared to an electric charge displacement field of an individual antigen or receptor, wherein the biomolecules in the electric charge displacement field of the plurality of antigens or receptors which are coupled in phase couple to the antigens or receptors specifically and rapidly.

2. The method as claimed in claim 1, wherein the biomolecules each contain at least two alpha helices or two beta sheets.

3. The method as claimed in claim 1, wherein antibodies which specifically bind to the biomolecules are used as the antigens or receptors.

4. The method as claimed in claim 1, wherein a liquid layer containing the biomolecules is formed above the substrate with the plurality of antigens or receptors which are coupled in phase, wherein the liquid layer has a layer thickness up to a maximum layer thickness at which all or the predominant fraction of the biomolecules in the liquid couple to the antigens or receptors.

5. The method as claimed in claim 1, wherein biomolecules are present in an ambient air or respiratory air aerosol, wherein the ambient air or respiratory air aerosol is guided over the substrate with the plurality of antigens or receptors which are coupled in phase, and wherein the temperature of the substrate with the plurality of antigens or receptors which are coupled in phase is controlled such that condensation of the ambient air or respiratory air aerosol occurs on the substrate.

6. The method as claimed in claim 1, wherein method is used to quantitatively determine and/or to separate the biomolecules, comprising neurotransmitters, interleukins, chemokines, enzymes, antibodies, viruses, bacteria and/or cells, from circulating blood, from a cerebrospinal fluid or from respiratory air from a human being or animal in vitro.

7. The method as claimed in claim 1, wherein the biomolecules are in a liquid or in an ambient air or respiratory air aerosol which is guided in a defined continuous flow process or throughflow process over the substrate with the plurality of antigens or receptors which are coupled in phase.

8. The method as claimed in claim 1, wherein an oxidizing material comprising silicon or aluminum is used as the substrate and blocking of regions of the substrate which are not occupied by antigens or receptors is carried out by means of oxidation or by means of other organic or inorganic materials.

9. The method as claimed in claim 1, wherein regions of the substrate which are not occupied by antigens or receptors are oxidized and an oxide layer which is formed thereby is rendered hydrophobic by a natural or synthetic coating.

10. The method as claimed in claim 1, wherein a linear or elliptically polarized laser beam is directed onto the substrate with the plurality of antigens or receptors which are coupled in phase with the coupled biomolecules and a laser beam which is reflected therefrom is detected in a detector.

11. The method as claimed in claim 10, wherein a camera is used as the detector.

12. The method as claimed in claim 10, wherein scattered light produced from the biomolecules coupled to the antigens or receptors is detected and analyzed for the quantitative determination of the number of coupled and therefore separated biomolecules.

13. The method as claimed in claim 10, wherein the substrate, with the plurality of antigens or receptors which are coupled in phase, is used multiple times for the quantitative determination and/or separation of biomolecules from different liquids or ambient air or respiratory air aerosols until a relationship between the number of biomolecules coupled to the antigens or receptors and the scattered light produced therefrom is no longer linear.

14. The method as claimed in claim 10, wherein for successive quantitative determinations and/or separations of biomolecules using the same substrate and the same plurality of antigens or receptors which are coupled in phase, the variation in the scattered light produced with respect to the previous quantitative determination and/or separation is analyzed analysed for the quantitative determination of the number of coupled and therefore separated biomolecules.

15. The method as claimed in claim 1, wherein the plurality of antigens or receptors is indirectly fixed to the substrate such that the plurality of antigens or receptors is applied to an elastic or rigid membrane comprised of a lipid membrane or a cell membrane or virus membrane with epitopes, and the membrane is floated onto the substrate on a layer of water.

16. The method as claimed in claim 1, further comprising a step for the qualitative assessment of a biomolecule load based on the quantitatively determined number of coupled biomolecules compared to a specified reference value.

17. An analytical means for the quantitative determination and/or separation of biomolecules, the analytical means comprising:
a substrate; and
a plurality of antigens or receptors each with at least one alpha helix or a beta sheet,
wherein the plurality of antigens or receptors is directly or indirectly fixed on the substrate so closely together that vibrations of the plurality of antigens or receptors are coupled together in phase via electric charges or mechanically,
wherein a larger electric charge displacement field of the plurality of antigens or receptors which are coupled in phase is formed compared to an electric charge displacement field of a single antigen or receptor,
wherein the larger electric charge displacement field of the plurality of antigens or receptors which are coupled in phase can be used to couple biomolecules to the antigens or receptors specifically and rapidly.

18. An analytical means as claimed in claim 17, wherein the antigens or receptors are antibodies which specifically bind to the biomolecules.

19. An analytical means as claimed in claim 17, wherein the temperature of the substrate with the plurality of antigens or receptors which are coupled in phase can be controlled such that an ambient air or respiratory air aerosol containing the biomolecules condenses on the substrate.

20. An analytical means as claimed in claim 17, wherein the substrate comprises an oxidizing material and regions of the substrate which are not occupied by antigens or receptors are occupied by means of oxidation or by another organic or inorganic material.

21. An analytical means as claimed in claim 17, wherein regions of the substrate which are not occupied by antigens or receptors have an oxide layer which is hydrophobic because of a natural or synthetic coating.

22. An analytical means as claimed in claim 17, wherein the plurality of antigens or receptors are indirectly fixed on the substrate such that the plurality of antigens or receptors is applied to an elastic or rigid membrane and the membrane is floated onto the substrate on a layer of water.

23. An analytical device for the quantitative determination and/or separation of biomolecules in a liquid or an ambient air or a respiratory air aerosol, wherein the analytical device comprises:
an analytical means as claimed in claim 17,
a continuous flow or throughflow device for guiding the liquid or the ambient air or respiratory air aerosol over the analytical means,
a laser beam decoupler for decoupling a linear or elliptically polarised laser beam onto the analytical means, and
a detector for detecting a laser beam reflected from the analytical means.

24. The analytical device as claimed in claim 23, wherein the analytical means is disposed in the analytical device so that the analytical means can be changed after a single use or after multiple uses.

25. The analytical device as claimed in claim 23, wherein the detector is a camera.

26. The analytical device as claimed in claim 23, further comprising an analysis unit which is equipped and configured to analyze scattered light produced from the biomolecules coupled to the antigens or receptors of the analytical means, which is detected by the detector for the quantitative determination of the number of coupled biomolecules.

27. The analytical device as claimed in claim 26, wherein the analysis unit is further equipped and configured to qualitatively assess a biomolecule load based on the quantitatively determined number of coupled biomolecules in comparison to a specified reference value.

28. The analytical device as claimed in claim 23, further comprising a temperature control unit which is equipped and configured to control the temperature of the analytical means so that an ambient air or respiratory air aerosol containing the biomolecules condenses on the analytical means.
